(19) United States
(12) Patent Application Publication
(10) Pub. No.: US 2016/0313430 A1
(43) Pub. Date: Oct. 27, 2016

(54) METHOD AND APPARATUS FOR QUANTITATIVE T1 DETERMINATION IN MAGNETIC RESONANCE IMAGING

(71) Applicant: Siemens Aktiengesellschaft, Muenchen (DE)

(72) Inventor: Marcel Dominik Nickel, Herzogenaurach (DE)

(73) Assignee: Siemens Aktiengesellschaft, Muenchen (DE)

(21) Appl. No.: 15/135,812

(22) Filed: Apr. 22, 2016

(30) Foreign Application Priority Data
Apr. 22, 2015 (DE) 102015207352.1

(51) Int. Cl. G01R 33/563; G01R 33/48; A61B 5/055; G01R 33/56

(52) U.S. Cl. CPC G01R 33/56316; G01R 33/5601; G01R 33/4828; G01R 33/56341; A61B 5/055

(57) ABSTRACT

In a method and magnetic resonance (MR) apparatus for quantitative T1 determination in MR imaging, MR data of the volume section are acquired depending on a contrast agent administered in the examined object, wherein the MR data of the volume section are acquired several times during various phases of the diffusion of the contrast agent in the volume section. First MR data of the volume section are acquired with a first sequence and second MR data of the volume section are acquired with a second sequence, wherein the first sequence is distinguished from the second sequence only by the flip angle of at least one RF pulse in the respective sequences and/or only by the repetition time of the respective sequences. Respective T1 values of each voxel of the volume section are determined depending on the first MR data and the second MR data.

### BACKGROUND OF THE INVENTION

1. Field of the Invention

The present invention concerns T1 determination, in particular, in so-called DCE-MR imaging (DCE=“Dynamic Contrast Enhanced”).

2. Description of the Prior Art

According to the prior art, DCE-MR imaging (dynamic contrast enhanced MR imaging) is performed with gradient echo sequences in order to create T1-weighted MR images for various phases of a contrast medium concentration. The results of this MR imaging are achieved by comparing the signal intensities during various phases of contrast medium concentration.

### SUMMARY OF THE INVENTION

An object of the present invention is to improve the quality of the results in DCE-MR imaging compared to the prior art.

Within the context of the present invention a method for quantitative T1 determination in MR imaging of a volume section of an examined object is provided as a function of a contrast agent. The method according to the invention comprises the following steps.

The inventive method begins with the administration of a contrast agent in the examined object. In this step, the contrast agent is injected into, for example, a blood vessel in the examined object (usually a person).

The next step in the inventive method is the acquisition of MR data of the volume section by the operation of an MR scanner. The MR data of the volume section are sampled or recorded several times during different diffusion phases of the contrast agent in the volume section. A diffusion phase of the contrast agent as used herein means both a time before the injection of the contrast agent as well as a time after the injection of the contrast agent. First MR data of the volume section are acquired in a first sequence and second MR data of the volume section are acquired in a second sequence. The first sequence is distinguished from the second sequence only by the flip angle or tilt angle, or only by the repetition time, or only by the flip angle and the repetition time.

The flip angle describes the angle by which magnetization is deflected by an RF pulse of the respective sequence. Acquisition of MR data can take place either only with the first and the second sequence, or in addition to the first and the second sequence with additional sequences.

In other words, MR data are acquired with at least two sequences which can be, but need not be, distinguished from each other by the flip angle and/or the repetition time, respectively. It is important that at least two of the sequences used (namely, at least the first sequence and the second sequence) differ from each other with regard to the flip angle that is used, and/or with regard to the repetition time that is used.

The method concludes with the determination of T1 values in a computer, voxel-by-voxel of the volume section, depending on the first MR data and the second MR data, and making the determined T1 value available in electronic form from the computer, such as in a data file.

With dynamic equilibrium of magnetization (steady state), a detected MR signal S(x) can be calculated using the following equation (1).

$$S ( x ) = ρ ( x ) \frac{1 - {e}^{- \frac{{T}_{R}}{{T}_{1} ( x )}}}{1 - cos ( α ( x ) ) {e}^{- \frac{{T}_{R}}{{T}_{1} ( x )}}} sin ( α ( x ) ) \qquad ( 1 )$$

wherein ρ(x) is the proton density, TR the repetition time, T1(x) the T1 value or relaxation time and α(x) the flip angle. The respective voxel is designated by x.

If an MR signal is now detected for the same voxel using different flip angles and/or using different repetition times, both the proton density ρ(x) and the T1 value T1(x) can be calculated. Thus, if at least two MR signals, each with different flip angles and/or with different repetition times, are detected for each voxel of the volume section, the T1 value and/or the relaxation time can be determined for each voxel. On the basis of this quantitative T1 value per voxel, an MR image of the volume section can then be created.

Compared with the prior art, this quantitative MR imaging according to the invention in which only T1-weighted MR images are generated has significant advantages with regard to repeatability and comparability.

In an embodiment of the invention, before the administration of the contrast agent the volume section is only sampled at least once (completely) with the first sequence and at least once (completely) with the second sequence, while for phases of diffusion of the contrast agent after the administration of the contrast agent, the volume section is only sampled once in each case (in particular, with the same sequence). As used herein, complete sampling of the volume section means sampling of k-space corresponding to the volume section, which is sufficient to create one MR signal for each voxel of the volume section.

As noted above, both the proton density and the T1 value per voxel can be determined on the basis of the first and second MR data (which, for example, are acquired before the administration of the contrast agent). Provided that the proton density per voxel is constant, in other phases of the diffusion of the contrast agent it then suffices to only determine one MR signal per voxel with which, for example, the T1 value per voxel can then be calculated on the basis of the equation (1).

The flip angle of at least one sequence can be selected according to the invention such that the signal-to-noise ratio of the generated MR image is optimized. The so-called Ernst angle of water, for example, can be used for this purpose.

The first and the second sequences can be selected such that the volume section is sampled with the same resolution both in the first sequence and in the second sequence. By this procedure, an MR image created on the basis of the first sequence can be brought into registration with an MR image created on the basis of the second sequence (described below). Furthermore, methods for noise suppression that require the same MR image resolution can be used.

According to the invention, however, it is also possible to sample the volume section (completely) with the first sequence during several phases of the diffusion of the contrast agent, for example, during each phase, wherein the first MR data are acquired, and in addition, to sample the volume section (completely) with the second sequence, wherein the second MR data are acquired.

As a result, the proton density and the T1 value per voxel can be determined during each phase on the basis of the first and second MR data, as a result of which the accuracy in particular of determining the T1 values can be increased.

According to the invention it is also possible to acquire the MR data not in each phase but, for example, only in two, three, four or more than four phases using the first and the second sequence. In the other phases in which the MR data is acquired without using the first and the second sequence, the MR data can either not be acquired at all or using another sequence. The sequences used (in other words, the first, second and other sequences) can be distinguished with regard to their flip angle and/or their repetition time. However, it is also possible for the other sequences to have the same flip angle and the same repetition time as the first and/or the second sequence. For this reason it is also possible that in each phase in which MR data is acquired, the MR data are acquired with another flip angle and/or with another repetition time.

In a further embodiment of the invention, an MR image is reconstructed for each phase of diffusion of the contrast agent in which MR data is acquired. These MR images generated for each phase are registered with each other in order to determine the T1 values per voxel on the basis of the registered MR images.

Registration can ensure that a pixel (T1 value) of an MR image which corresponds to a particular voxel of the volume section is assigned to the pixel (T1 value) in another MR image which corresponds to the same voxel of the volume section. If the proton density for the voxel for one MR image is known, this proton density can thus also be assumed for the associated voxel in the other MR image in order to determine the T1 value of the corresponding voxel in the other MR image depending on this proton density. In other words, registration ensures that a particular voxel of the volume section is assigned to the correct pixel in that registered MR image.

In another embodiment of the invention, the method described can be combined with the so-called Dixon method. To this end, the first (second) MR data are acquired several times in order to acquire the part of the first (second) MR signal for each voxel corresponding to a predetermined chemical component in accordance with the Dixon method. The first (second) MR signal is the MR signal of the corresponding voxel ascertained from the measurement of the first (second) MR data. On the basis of the first and the second MR signals of the predetermined chemical component in the respective voxel, the T1 value of this predetermined chemical component can then be determined in the respective voxel. When determining the T1 value of the predetermined chemical component, it is assumed that this T1 value and/or the proton density of the predetermined chemical component in the voxel are constant.

The predetermined chemical component may be fat, silicon, water or hyperpolarized ¹³C.

If the predetermined chemical component is fat or silicon, for example, the T1 value of the fat or the silicon in the respective voxel should not change. I.e. the T1 value of the fat or the silicon remains constant regardless of the concentration of the contrast agent because the contrast agent does not diffuse in fat or silicon.

If (only) the T1 value of fat or silicon is assumed to be constant, the determination of the T1 value for other voxels (in which neither fat nor silicon dominates) can also be optimized as a result of this assumption. For example, the constancy of the T1 value for fat and silicon voxels may be regarded as a condition to be met, which must be observed in a fitting process to determine the T1 values for all voxels for all phases.

The Dixon method is understood to mean a method in which the (first or second) MR data are acquired several times (i.e. the volume section is sampled several times (completely) in order to acquire the corresponding (first or second) MR data) in order to determine the MR signal of the predetermined chemical component on the basis of linking the MR signals recorded for each measurement. In order, for example, to obtain the MR signal of a first chemical component (e.g. fat) on condition that in addition to the first chemical component essentially only one additional second chemical component (e.g. water) is present in the corresponding voxel, a sequence is used in which the MR signal of the first component and the MR signal of the second component are in phase, and a further sequence is used in which the MR signal of the first component and the MR signal of the second component are displaced by 180°. In the in-phase image I₀=K₁+K₂ is valid for each pixel and in the 180°-displaced image or out-of-phase image I₁=K1−K2. Then the MR signal of the first component for each pixel can be calculated by K₁=½(I₀+I₁) and the MR signal of the second component for each pixel by K₂=½(I₀−I₁).

By using the Dixon method, the T1 value can be determined for more than one chemical component within each voxel. Based on certain conditions which only apply to certain chemical components (for example, the constancy of the T1 value for fat and silicon), the signal-to-noise ratio, for example, can then be improved when determining the T1 values for all voxels.

Generally speaking, the present invention can be executed using different kinds of sequences (e.g. spin-echo sequences), the use of gradient echo sequences being preferred, however.

With the present invention the problem of so-called B1 inhomogeneity can also be moderated at least. With B1 inhomogeneity a distinction is drawn between B₁⁺ inhomogeneity and B₁⁻ inhomogeneity.

On account of B₁⁺ inhomogeneity, which in particular describes the inhomogeneity of the B1 field during absorption of the RF pulse, the desired flip angle does not correspond to the actual flip angle α(x) in the voxel x. While, in particular, the fat content and the water content is determined for each voxel and while it is assumed that the T1 value is constant in those voxels in which the fat content dominates, on the basis of the aforementioned equation (1), for example, the actual flip angle α(x) can be determined for the fat voxels (in which the fat content dominates). By means of interpolation or extrapolation of the actual flip angle of the fat voxel, the actual flip angle can then be determined for all the voxels of the volume section.

The non-uniform sensitivity of the receiving coil(s) is described by the B₁⁻ inhomogeneity. According to the invention, this B₁⁻ inhomogeneity can be corrected by assuming that in those voxels in which the fat content dominates, both the proton density and the T1 value in the respective fat voxel (in which the fat content dominates) is constant. In turn, (as in the correction according to the invention of B₁⁺ inhomogeneity) by inter/extrapolating the proton density and the T1 value of the fat voxel to all voxels, the T1 value can ultimately be determined with great accuracy for all voxels.

Furthermore, for each voxel of the volume section it is possible to determine whether the respective voxel is inside the examined object or outside the examined object. For those voxels which are inside the examined object, it is assumed (regardless of the chemical component which dominates inside the respective voxel) that the proton density of the respective voxel is constant. This assumption also means that it is possible to determine the T1 value for all the voxels with great accuracy.

The T1 value describes the mono-exponential relaxation of longitudinal magnetization of a species or chemical component in the respective voxel. If two species in the same voxel have a significant share, relaxation corresponds to the total of two exponential functions (also referred to as bi-exponential) so that strictly speaking, in this case the T1 value is no longer defined because the signal path no longer corresponds to that of an individual species and/or is no longer mono-exponential. In this case, in the present invention (if two (or more) species in the same voxel have a significant share) a mono-exponential signal path is nevertheless adjusted to the detected signal path so that a so-called effective T1 value is determined. The T1 value determined in this way lies between the T1 values of both species, having a tendency to the higher T1 value (of both species).

The present invention also encompasses a magnetic resonance apparatus for quantitative T1 determination in MR imaging of a predetermined volume section of an examined object. The magnetic resonance apparatus has an MR data acquisition scanner that has a basic field magnet, a gradient coil arrangement, at least one RF antenna and a control computer for control of the gradient coil arrangement and the at least one RF antenna for the reception of detected signals from the RF antenna(e) and for evaluation of the measuring signals and for creation of the MR images. The magnetic resonance apparatus is designed such that, depending on the administration of a contrast agent in the examined object, the magnetic resonance system acquires MR data of the volume section in order to sample (acquire MR data from) the volume section several times during various phases of diffusion of the contrast agent in the volume section. The magnetic resonance system acquires first MR data of the volume section with a first sequence and acquires second MR data of the volume section with a second sequence. The first sequence is from the second sequence distinguished by only the flip angle and/or only the repetition time of the respective sequences. The magnetic resonance apparatus is designed to determine T1 values for each voxel of the volume section depending on the first MR data and the second MR data.

The advantages of the magnetic resonance apparatus according to the invention essentially correspond to the advantages of the method according to the invention explained in detail above.

The present invention also encompasses a non-transitory, computer-readable data storage medium that can be loaded into a memory of a programmable controller or computer of a magnetic resonance apparatus. The storage mediums is encoded with programming instructions (code) that cause all or several previously described embodiments of the method according to the invention to be implemented when the code is executed in the controller or computer of the magnetic resonance apparatus. The programming instructions may require further programming means, e.g. libraries and auxiliary functions, to realize the embodiments of the method. The code may be a source code (e.g. C++) which still has to be compiled (translated) and linked or which only needs to be interpreted, or an executable software code that only needs to be loaded into the corresponding computer or controller for execution.

The electronically readable data carrier can be, e.g. a DVD, a magnetic tape, a hard disk or a USB stick on which electronically readable control information software (cf. above), is stored.

The present invention is suitable for dynamic contrast-agent enhanced MR imaging in which measurements or MR images are assessed in qualitative terms by comparing the signal intensities of MR images (i.e. the T1 values for each voxel) between various phases of diffusion of the contrast agent. The present invention thus enables all dynamic MR imaging to be transferred from T1-weighted MR imaging to quantitative T1 imaging.

### BRIEF DESCRIPTION OF THE DRAWINGS

FIG. 1 is a block diagram of a magnetic resonance apparatus according to the invention.

FIG. 2 shows a gradient echo sequence according to the invention.

FIG. 3 is a flowchart of a method according to the invention.

### DESCRIPTION OF THE PREFERRED EMBODIMENTS

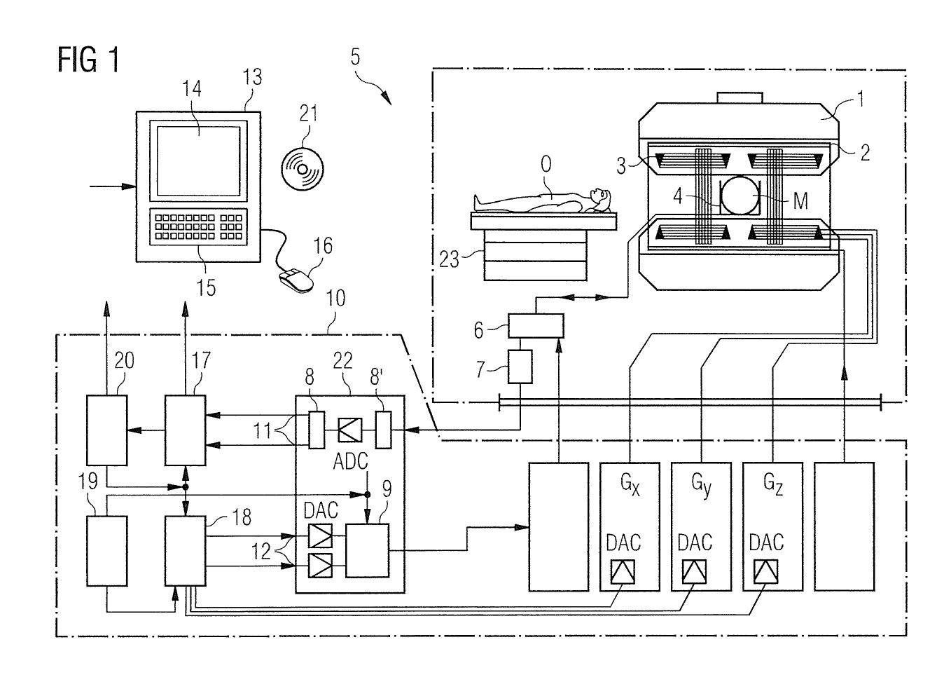

FIG. 1 is a block diagram of a magnetic resonance apparatus that has an MR data acquisition scanner 5 according to the invention (a magnetic resonance imaging or tomography apparatus). The MR data acquisition scanner 5 has a basic field magnet 1 that generates a temporally constant strong basic magnetic field for polarization or alignment of the nuclear spin in an examination area of an object O, such as a part of a human body situated for examination in the MR scanner, lying on a table 23. The high homogeneity of the basic magnetic field that is necessary for a nuclear spin resonance measurement is defined in a typically spherical measuring volume M, in which the volume section of the human body for examination is arranged. To support homogeneity requirements, and in particular, to eliminate temporally invariable influences, shim plates made of ferromagnetic material are attached at appropriate points. Temporally variable influences are eliminated by shim coils 2.

A cylindrical gradient field system (gradient coil system 3) having three sub-windings is present in the basic field magnet 1. Each sub-winding is supplied with current by an amplifier so as to generate a linear (and temporally variable) gradient field in a respective direction of a Cartesian coordinate system. The first sub-winding of the gradient coil system 3 generates a gradient Gₓ in the x-direction, the second sub-winding a gradient G_{y} in the y-direction, and the third sub-winding a gradient G_{z} in the z-direction. The amplifier has a digital-to-analog converter that is controlled by a sequence controller 18 in order to generate gradient pulses in an appropriately timed fashion.

Within the gradient coil system 3 at least one radio-frequency (RF) antenna is situated, which converts RF pulses emitted by an RF power amplifier into a magnetic alternating field so as to excite the nuclei and thereby cause the nuclear spin of the object for examination O or the area of the object for examination O to deviate ((by an amount called the flip angle) from the alignment produced by the basic magnetic field. Each RF antenna 4 is formed by one or more RF transmitter coils and one or more RF receiver coils in the form of a toroidal, preferably linear or matrix-shaped, arrangement of component coils. The alternating field originating from the precessing nuclear spins, i.e. usually nuclear spin echo signals triggered by a pulse sequence of one or more RF pulses and one or more gradient pulses, is also converted into a voltage (measured signal) by the RF receiver coils of the respective RF antenna 4, and is fed to an RF reception channel 8 of an RF system 22 via an amplifier 7. The RF system 22, which is part of a control computer 10 of the magnetic resonance system 5, furthermore has a transmission channel 9 in which RF pulses for the excitation of magnetic nuclear resonance are generated. The respective RF pulses are produced as the result of a sequence of complex numbers on the basis of a pulse sequence predefined by the system computer 20 in the sequence controller 18. This numerical sequence is fed as a real part and an imaginary part via a respective inputs 12 to a digital-to-analog converter in the RF system 22, and from this to a transmission channel 9. In the transmission channel 9 the pulse sequences are modulated to a RF carrier signal with a base frequency that corresponds to the resonance frequency of the nuclear spins in the measuring volume.

Switching from transmit mode to receive mode takes place via a duplexer 6. The RF transmitter coils of the RF antenna(e) 4 emit/s RF pulses into the measuring volume M to excite the nuclear spins, and resultant echo signals are sampled via RF-receiving coil(s). The nuclear resonance signals thus obtained are phase-sensitively demodulated in the reception channel 8′ (first demodulator) of the RF system 22 to an intermediate frequency, digitized in the analog-to-digital converter (ADC), and emitted via the output 11. In addition, this signal is demodulated to the frequency 0. Demodulation to a frequency 0 and separation into a real part and imaginary part take place in a second demodulator 8 after digitization in the digital domain. An MR image is reconstructed by an image processor 17 from the measurement data obtained in such a way via an output 11. The administration of the measurement data, the image data and the control programs takes place via the system computer 20. Due to a specification with control programs, the sequence controller 18 monitors the generation of the respective pulse sequences desired and the corresponding sampling of k-space. In particular, the sequence controller 18 controls the correctly timed switching of the gradients, the emission of RF pulses with defined phase amplitude and the reception of nuclear resonance signals. A synthesizer 19 provides the time base for the RF system 22 and the sequence controller 18. The selection of corresponding control programs for the generation of an MR image that, for example, are stored on a DVD 21, and the display of the generated MR image takes place via a terminal 13 having a keyboard 15, a mouse 16 and a screen 14.

The system computer 20 in the magnetic resonance system 5 according to the invention is designed to sample the entire volume section of the examined object with a first sequence to acquire first MR data, and to sample the entire volume section with a second sequence to acquire second MR data. The first sequence is from the second sequence only distinguished by the flip angle and/or only by the repetition time of the respective sequences. As a result, the image processor 17 of the magnetic resonance system 5 according to the invention can determine the T1 value per voxel of the volume section depending on the first MR data and the second MR data in order to reconstruct the MR image on the basis of the T1 values.

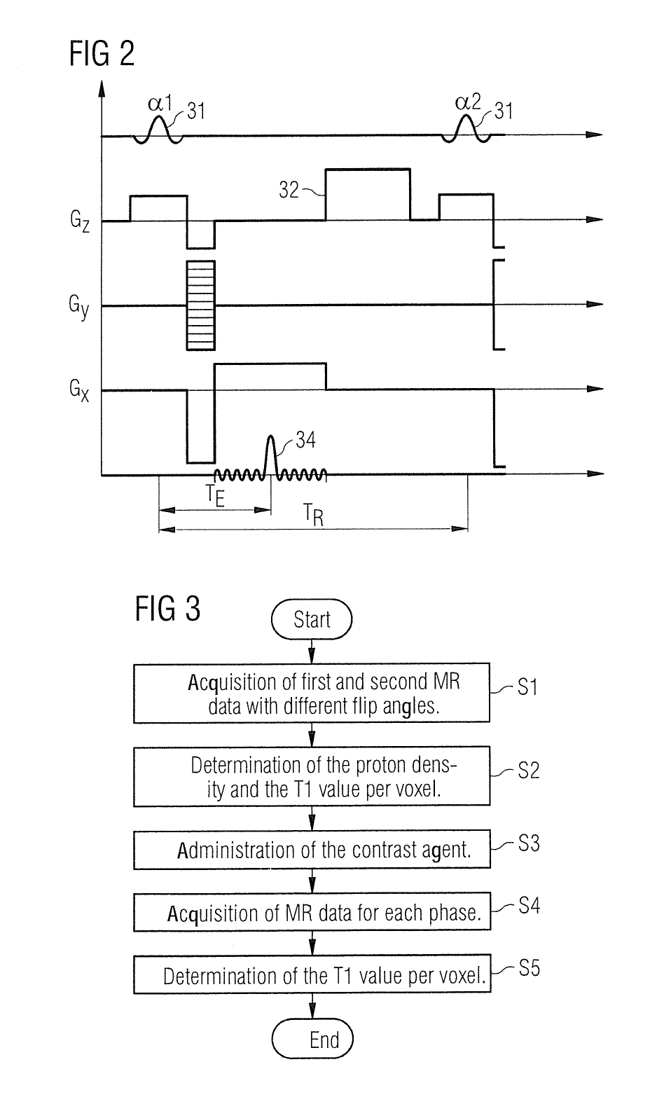

FIG. 2 shows a gradient-echo sequence according to the invention. Initially, an RF excitation pulse 31 with a flip angle α₁ is switched while a slice-selection gradient Gz is activated at the same time. By changing the polarity of the slice-selection gradient Gz after the RF-excitation pulse 31, the phase response which has arisen during excitation is reversed. At the same time, the signals from spins are encoded by switching the frequency encoding gradient Gx (this part of the frequency encoding gradient Gx is also known as a rewinder). The phase encoding gradient Gy, likewise switched after the RF excitation pulse 31, is for spatial encoding. By changing the polarity of the frequency encoding gradient Gx, the previously encoded spins are brought back into phase or rephased, resulting in the gradient echo 34. While the frequency encoding gradient Gx (in FIG. 2) has its positive polarity, measurement data are acquired and are entered into a k-space line in the x-direction.

After the frequency encoding gradient Gx or after the acquisition of the measurement data, a spoiler gradient 32 is switched to eliminate transverse magnetization, also known as a gradient-echo sequence with spoiler. After this spoiler gradient 32, the next RF excitation pulse 31 is switched, resulting in the start of a further period of the gradient-echo sequence. However, this next RF excitation pulse 31 has a different flip angle α₂ compared with the previous RF excitation pulse.

The echo time TE is measured from the RF excitation pulse 31 until the gradient echo 34, which occurs chronologically in the middle of the positive part (according to FIG. 2) of the frequency encoding gradient Gx. The repetition time or repetition time TR determines the time interval between two temporally adjacent RF excitation pulses 31.

FIG. 3 is a flowchart of an embodiment of the method according to the invention for quantitative T1 determination.

In step S1, before the administration of a contrast agent (see step S3), the volume section of the examined object is sampled (completely) using a first flip angle, first MR data being acquired. In this step S1, the volume section is sampled again (completely) using a second flip angle different from the first flip angle, second MR data being acquired. On the basis of the first and second MR data, in step S2 the proton density and the repetition time or the T1 value per voxel of the volume section can then be determined, for example, on the basis of the aforementioned equation (1).

After step S3, in which the contrast agent is administered, respective MR data are acquired for each of various phases, each of which represents a certain diffusion stage of the contrast agent in the volume section of the examined object. In step S4 the volume section is (completely) sampled for each phase in order to acquire the MR data corresponding to the respective phase. If it is assumed that the proton density determined in step S2 is constant for each voxel throughout all the phases, one MR signal per voxel is sufficient to determine the T1 value per voxel of the volume section, for example, on the basis of the equation (1) in step S5.

Although modifications and changes may be suggested by those skilled in the art, it is the intention of the inventor to embody within the patent warranted hereon all changes and modifications as reasonably and properly come within the scope of his contribution to the art.

I claim as my invention:

1. A method for quantitative T1 determination in magnetic resonance (MR) imaging, comprising:
administering a contrast agent to an examination subject;
operating an MR data acquisition scanner, while the examination subject is situated therein, to acquire MR data from a selected volume of the examination subject while the contrast agent proceeds through a plurality of diffusion phases in said selected volume;
operating said MR data acquisition scanner to acquire said MR data by executing a first data acquisition sequence with which a first set of MR data is acquired from the selected volume and by executing a second data acquisition sequence with which a second set of MR data is acquired from the selected volume, said first and second sequences each comprising at least one radio-frequency (RF) pulse having a flip angle associated therewith, and each having a repetition time, and said first and second sequences differing from each other by a difference selected from the group consisting of only the flip angle associated with the at least one RF pulse, only the repetition time, and only the flip angle of the at least RF pulse and the repetition time;
providing said first and second sets of MR data to a computer and, in said computer, determining respective T1 values for each voxel of said selected volume from said first set of MR data and said second set of MR data; and
making the determined T1 values per voxel available from the computer in electronic form as a data file.

2. A method as claimed in claim 1 comprising acquiring MR data only from said volume section with said first data acquisition sequence and said second data acquisition sequence before said contrast agent is administered.

3. A method as claimed in claim 1 comprising acquiring said MR data from said selected volume with said first data acquisition sequence and with said second data acquisition sequence during each of diffusion phases.

4. A method as claimed in claim 1 comprising, in said computer, using MR data acquired during a same diffusion phase, among said plurality of diffusion phases from said first set of MR data and said second set of MR data to determine, in addition to said T1 value, a proton density for each voxel, and assuming that the determined proton density is constant throughout said selected volume, and determining said T1 value per voxel from a part of said selected volume from which MR data are acquired with only one of said first or second sequences, using the determined proton density per voxel for said same diffusion phase.

5. A method as claimed in claim 1 comprising:
in an image processor, reconstructing respective MR images of said volume section from the acquired MR data; and
bringing said MR images into registration with each other in said computer before determining the respective T1 values per voxel.

6. A method as claimed in claim 1 comprising:
also operating said MR data acquisition scanner according to a Dixon method to determine an extent to which a predetermined chemical component is present in each voxel of said selected volume;
determining the T1 value of the predetermined chemical component per voxel; and
determining said T1 value per voxel of the predetermined chemical component in said computer by assuming, in said computer, that at least one of said T1 value and a proton density of the chemical component in the voxel remain constant.

7. A method as claimed in claim 6 comprising selecting said chemical component from the group consisting of fat, silicon, water and hyperpolarized ¹³C.

8. A method as claimed in claim 1 comprising determining the respective T1 values per voxel by assuming, in said computer, that each voxel has a same proton density for each diffusion phase.

9. A method as claimed in claim 1 comprising operating said MR data acquisition scanner with each of said first data acquisition sequence and said second data acquisition sequence being a gradient-echo sequence.

10. A magnetic resonance (MR) apparatus comprising:
an MR data acquisition scanner;
a contrast agent injector that administers a contrast agent to an examination subject;
a control computer configured to operate the MR data acquisition scanner, while the examination subject is situated therein, to acquire MR data from a selected volume of the examination subject while the contrast agent proceeds through a plurality of diffusion phases in said selected volume;
said control computer being configured to operate said MR data acquisition scanner to acquire said MR data by executing a first data acquisition sequence with which a first set of MR data is acquired from the selected volume and by executing a second data acquisition sequence with which a second set of MR data is acquired from the selected volume, said first and second sequences each comprising at least one radio-frequency (RF) pulse having a flip angle associated therewith, and each having a repetition time, and said first and second sequences differing from each other by a difference selected from the group consisting of only the flip angle associated with the at least one RF pulse, only the repetition time, and only the flip angle of the at least RF pulse and the repetition time;
a processing computer provided with said first and second sets of MR data, said processing computer being configured to determine respective T1 values for each voxel of said selected volume from said first set of MR data and said second set of MR data; and
said processing computer being configured to make the determined T1 values per voxel available from the processing computer in electronic form as a data file.

11. A non-transitory, computer-readable data storage medium encoded with programming instructions, said storage medium being loaded into a control and processing computer of a magnetic resonance (MR) apparatus that comprises an MR data acquisition scanner and a contrast agent injector that administers a contrast agent to an examination subject, and said programming instructions causing said control and evaluation computer to:
operate the MR data acquisition scanner, while the examination subject is situated therein, to acquire MR data from a selected volume of the examination subject while the contrast agent proceeds through a plurality of diffusion phases in said selected volume;
operate said MR data acquisition scanner to acquire said MR data by executing a first data acquisition sequence with which a first set of MR data is acquired from the selected volume and by executing a second data acquisition sequence with which a second set of MR data is acquired from the selected volume, said first and second sequences each comprising at least one radio-frequency (RF) pulse having a flip angle associated therewith, and each having a repetition time, and said first and second sequences differing from each other by a difference selected from the group consisting of only the flip angle associated with the at least one RF pulse, only the repetition time, and only the flip angle of the at least RF pulse and the repetition time;
determine respective T1 values for each voxel of said selected volume from said first set of MR data and said second set of MR data; and
make the determined T1 values per voxel available from the control and evaluation computer in electronic form as a data file.
